(19) United States
(12) Patent Application Publication
(10) Pub. No.: US 2009/0281435 A1
(43) Pub. Date: Nov. 12, 2009

(54) METHOD AND APPARATUS FOR ROBUST HEART RATE SENSING

(75) Inventors: Mohamed I. Ahmed, Glendale Heights, IL (US); Mark W. Cholewczynski, Wheaton, IL (US); Faisal Ishtiaq, Chicago, IL (US); Fazlur M. Rahman, Niles, IL (US)

Correspondence Address: MOTOROLA, INC., 1303 EAST ALGONQUIN ROAD, IL01/3RD, SCHAUMBURG, IL, 60196, US

(73) Assignee: MOTOROLA, INC., Schaumburg, IL (US)

(21) Appl. No.: 12/116,510

(22) Filed: May 7, 2008

(51) Int. Cl. A61B 5/024

(52) U.S. Cl. 600/502; 600/508

(57) ABSTRACT

A heart rate sensing system (300) includes a light source (112, 310, 1008, 1110) a light detector (114, 1010, 1112) and a pressure sensor (116, 1012, 1114) held by a compressible comformable resilient pad (110, 1006, 1108) against a wearers body (202). A signal from the pressure sensor is used to alter the amplitude of a signal detected by the light source in order to reduce motion artifacts. The system can be incorporated into an article, such as an ear cuff (100), an audio headset (1000) or a set of headphones (1100), that is suitable for use by an active user.

### FIELD OF THE INVENTION

The present invention relates generally to sensor signal processing systems.

### BACKGROUND

Certain systems that use sensors are prone to “sensor dropout” a condition in which the sensor temporarily looses the ability to sense and an invalid signal or no signal at all is produced by the sensor. Systems that use sensors can also suffer from noise contamination of the signals produced by the sensors. The noise contamination may arise from a wide variety of phenomenon depending on the type of sensor.

One type of sensor system that is prone to sensor dropout and noise contamination is an optical heart rate monitor system. Using optical sensors to detect heart rates is called photoplethysmography. An optical heart rate monitor includes an optical radiation (e.g., visible or infra-red light) emitter (e.g., Light Emitting Diode or LED) and an optical radiation detector (e.g., a photodiode) held in a mechanical attachment mechanism proximate some extremity of a person's body (e.g., a finger or ear lobe). Optical heart rate monitor systems are distinguished from electrical heart rate monitor systems which require electrodes that must be adhered to the user, grasped by the user or incorporated into an article (e.g., chest strap) that is worn by the user. Unfortunately, optical heart rate sensing systems are prone to motion induced sensor dropout and noise contamination. This is particularly disadvantageous for applications of heart rate monitors where the user is expected to be mobile, for example heart rate monitors for athletes or fitness enthusiasts or heart rate monitors for ambulatory patients.

A general issue regarding optical heart rate monitors is that the provisions for holding them proximate the users' bodies are not ideal for applications where the user is expected to be active. For optical heart rate monitors perhaps the most common form factor resembles a clothes pin that clips to the user's ear. While suitable for a bed-ridden patients, the dangling mass of this design makes it unsuitable for use by moderately active people or people who are exercising. It would be desirable to have designs that are more suitable for use while exercising. As alluded to above movement in the course of exercise would also tend to cause signal degradation so this also needs to be addressed.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 is an isometric view of an ear cuff that incorporates an optical heart rate sensing system according to an embodiment of the invention;

FIG. 2 shows the ear cuff shown in FIG. 1 in position on a user's ear;

FIG. 3 is a functional block diagram of the heart rate sensing system used in the ear cuff shown in FIG. 1 or in other optical heart rate monitors according to embodiments of the invention;

FIG. 4 is a graph including three signal plots for signals at different stages in an optical heart rate sensing system according to an embodiment of the invention;

FIG. 5 is a graph including a plot of a partially processed signal in an optical heart rate detection system along with points identifying peaks associated with individual heart beats that have been detected by a peak detector;

FIG. 6 is a more detailed view of a portion of the heart rate detecting system shown in FIG. 3 according to an embodiment of the invention;

FIG. 7 is a schematic representation showing how a set of counts stored in a FIFO buffer shown in FIG. 7 are aggregated hierarchically by a windowed averager of the system shown in FIG. 3 to produce a set of running averages over three different time scales;

FIG. 8 is a block diagram of a state machine of the system shown in FIG. 3;

FIG. 9 is partial functional block diagram of a part of the system shown in FIG. 3 according to an alternative embodiment of the invention;

FIG. 10 is an audio headset that includes an optical heart rate sensing system according to an embodiment of the invention;

FIG. 11 is a set of headphones that includes an optical heart rate sensing system according to an embodiment of the invention; and

FIG. 12 shows one of the headphones shown in FIG. 11 located proximate a user's ear.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to signal processing. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

It will be appreciated that embodiments of the invention described herein may be comprised of one or more conventional processors and unique stored program instructions that control the one or more processors to implement, in conjunction with certain non-processor circuits, some, most, or all of the functions of signal processing described herein. The non-processor circuits may include, but are not limited to, a radio receiver, a radio transmitter, signal drivers, clock circuits, power source circuits, and user input devices. As such, these functions may be interpreted as steps of a method to perform signal processing. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

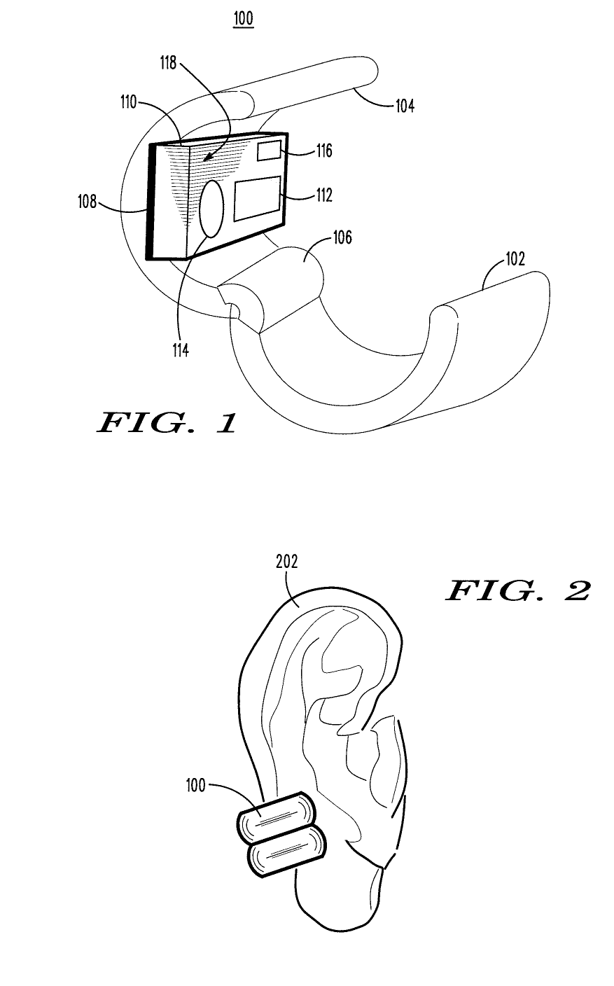

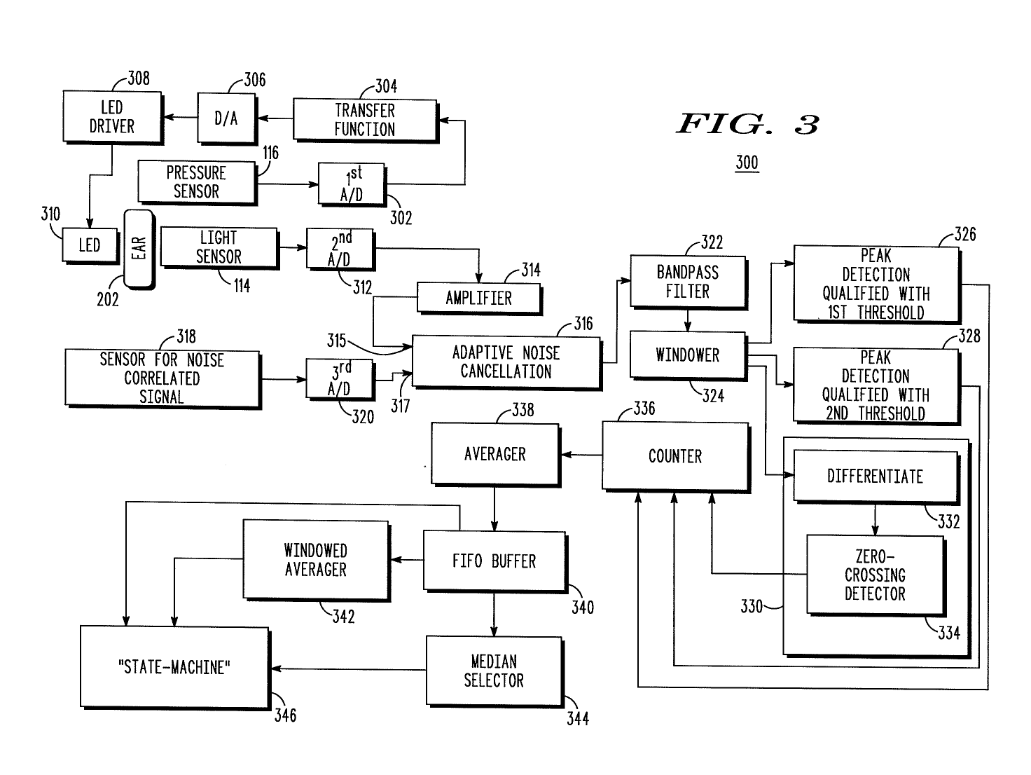

FIG. 1 is an isometric view of an ear cuff 100 that incorporates an optical heart rate sensing system 300 (FIG. 3) according to an embodiment of the invention. The cuff 100 is made up of a first arcuate half 102 and a second arcuate half 104 that are connected together by a hinge 106. A small printed circuit board 108 is mounted on the second arcuate half 104. A piece of compressible (comformable resilient) material 110 (e.g., foam rubber, epoxy gum) is affixed (e.g., with an adhesive) over the small printed circuit board 108. A light source e.g., a Light Emitting Diode (LED) 112, a light sensor, e.g., a photodiode 114 and a pressure sensor 116 are mounted at (or alternatively proximate) an outer surface 118 of the piece of compressible material 110.

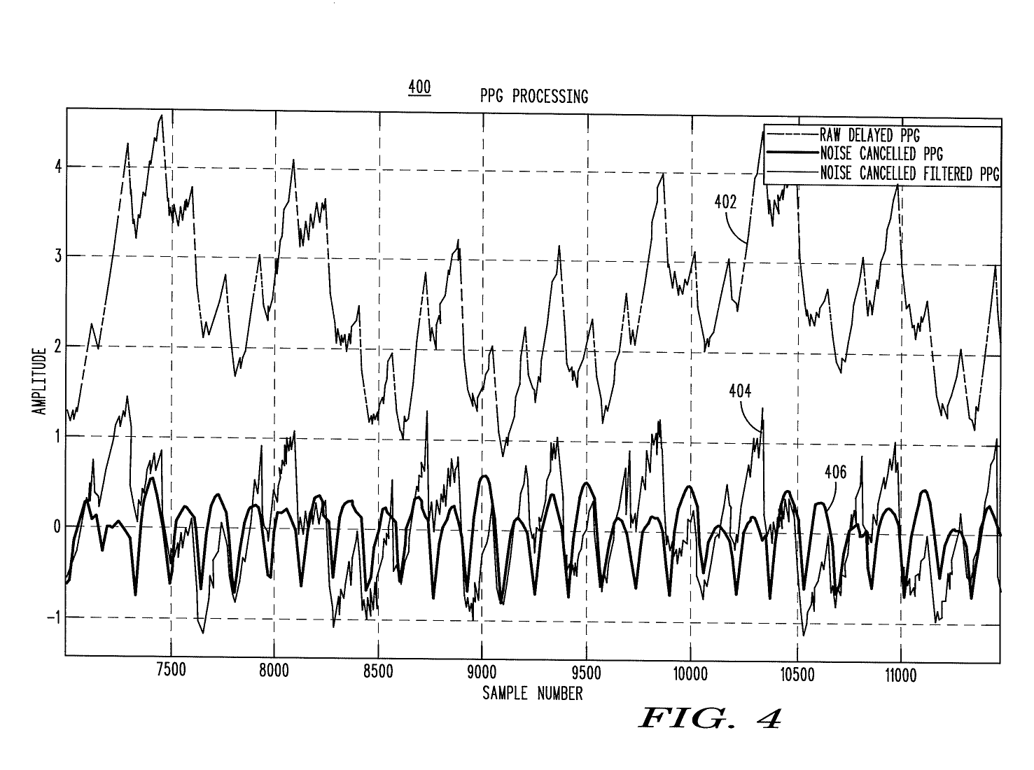

The light source 112 emits light that interacts with the living tissues of the wearer's earlobe in particular with blood pulsing through capillaries in response to the wearer's heart beat. The pulsing causes a correlated change in the optical properties (e.g., reflectance, transmittance, absorption) of the user's ear, which in turn causes a correlated change in the amount of light incident on the light sensor 114, and thus the wearer's heart beat is detected. Using light to detect a heartbeat is known as Photoplethysmography. The pressure sensor 116 is used to obtain a signal that is associated with motion artifacts that appear in the signal of the light sensor 114. The signal from the pressure sensor 116 is used to reduce the noise in the signal obtained from the light sensor 114. Signal processing using the signal from the pressure sensor 116 is more fully described below with reference to FIG. 3 and FIG. 4. Circuit components (not shown in FIG. 1) of a circuit for driving the light source 112 and processing signals from the light sensor 114 and the pressure sensor 116 are mounted on and connected by the small printed circuit board 108. The small printed circuit board 108 can also include a transceiver for communicating heart rate information to another device, e.g., the wearer's cellular telephony handset. FIG. 2 shows the ear cuff 100 shown in FIG. 1 in position on a user's ear 202. The ear cuff 100 format makes the heart rate sensing system 300 that it incorporates unobtrusive and inconspicuous.

FIG. 3 is a functional block diagram of the heart rate sensing system 300 used in the ear cuff 100 shown in FIG. 1 or in other optical heart rate monitors according to embodiments of the invention. As shown the pressure sensor 116 is coupled to a first analog-to-digital converter (A/D) 302 which supplies a digitized version of a pressure signal received from the pressure sensor through a transfer function (e.g., a time domain filter or lookup table) 304 to a digital-to-analog converter (D/A) 306. The D/A 306 in turn supplies an analog control signal to an LED driver 308 which drives an LED light source 310. Fluctuations in the pressure read by the pressure sensor 116 are correlated with signal artifacts (noise) in the light signal sensed by the light sensor 114 that are due to jostling of the ear cuff 100 (or other heart beat sensing devices). By means of the foregoing circuit, the intensity of light produced by the LED light source 310 is made to be a function of the pressure between the compressible material 110 and the wearer's ear 202. Accordingly, artifacts (noise) in the light signal sensed by the light sensor 114 that are due to jostling of the ear cuff 100 (or other heart beat sensing devices) caused by activity of the wearer are diminished. Optionally, an amplifier (not shown) can be interposed between the pressure sensor 116 and the first A/D 302.

As shown in FIG. 3 the light sensor 114 is coupled through a second A/D 312 and an amplifier 314 to a first input 315 of an adaptive noise canceller 316. A sensor for sensing a noise correlated signal 318 is coupled through a third A/D 320 to a second input 317 of the adaptive noise canceller 316. The sensor for sensing the noise correlated signal 318 can for example comprise an accelerometer. An accelerometer senses activity of the wearer of the ear cuff 100 (or other device including the heart rate detection system 300). Such activity can create motion artifacts (noise) in the light sensor 114 signal. Alternatively, in lieu of an accelerometer the pressure sensor 116 can be used as the sensor for sensing the noise correlated signal 318. The functioning of the adaptive noise canceller 316 is known to persons of ordinary skill in the signal processing art.

The adaptive noise canceller 316 is coupled to a bandpass filter 322. The bandpass filter 322 is designed to filter out frequency components that fall outside the range of normal heart beats, for example outside the range of 0.5 to 4.0 Hertz equivalent to a heart rate range of 30 to 240 beats per minute. Using the bandpass filter 322 helps clean up the signal by further reducing noise and facilitate heart beat detection.

FIG. 4 is a graph 400 including three signal plots 402, 404, 406 for signals at different stages in an optical heart rate sensing system according to an embodiment of the invention. A first plot 402 is of the signal produced by the light sensor 114, a second plot 404 is of the signal after processing by the adaptive noise canceller 316, and a third plot 406 is of the signal after the bandpass filter 322. It should be observed that both the adaptive noise canceller 316 and the bandpass filter 322 play a role in reducing noise in the signal. These plots are for a system without modulation of the light source according to the pressure sensor signal.

Referring again to FIG. 3, the bandpass filter 322 is coupled to a windower 324. The windower 324 breaks the signal up into a sequence of windows which are passed to a number of peak detectors 326, 238, 330. Although three peak detectors are shown in FIG. 3, alternatively a different number of peak detectors can be used.

A first peak detector 326 scans each window looking for peak signal samples that is signal samples that are greater than both the preceding and succeeding signal sample. This can be written as S_{K-1}<S_{K}>S_{K+1}. To satisfy the first peak detector 326, meeting foregoing inequality is a necessary but not sufficient condition. The first peak detector 326 can comprises a pair of comparators in order to test to foregoing inequality relations. In order to be identified as a valid peak a particular signal sample S_{K} must also exceed a threshold magnitude that is suitably scaled to the energy in the window being processed. This can be written as |S_{K}|>T*RMS where T is a preprogrammed threshold factor and RMS is a root means square measure of the energy in window. The threshold factor T is suitably in the range of 0.0 to 0.3 for a normalized signal whose range is from −1 to 1. Adding the threshold amplitude requirement allows the system 300 avoid construing low, e.g., near zero signal oscillations as actual heart beats. The first peak detector 326 can comprise a third comparator to verify the latter inequality. A second peak detector 328 is the same as the first peak detector 326 but uses a different value of the threshold factor T. For example, the first peak detector 326 can use a threshold factor of 0.05 and the second peak detector 328 a threshold factor of 0.3.

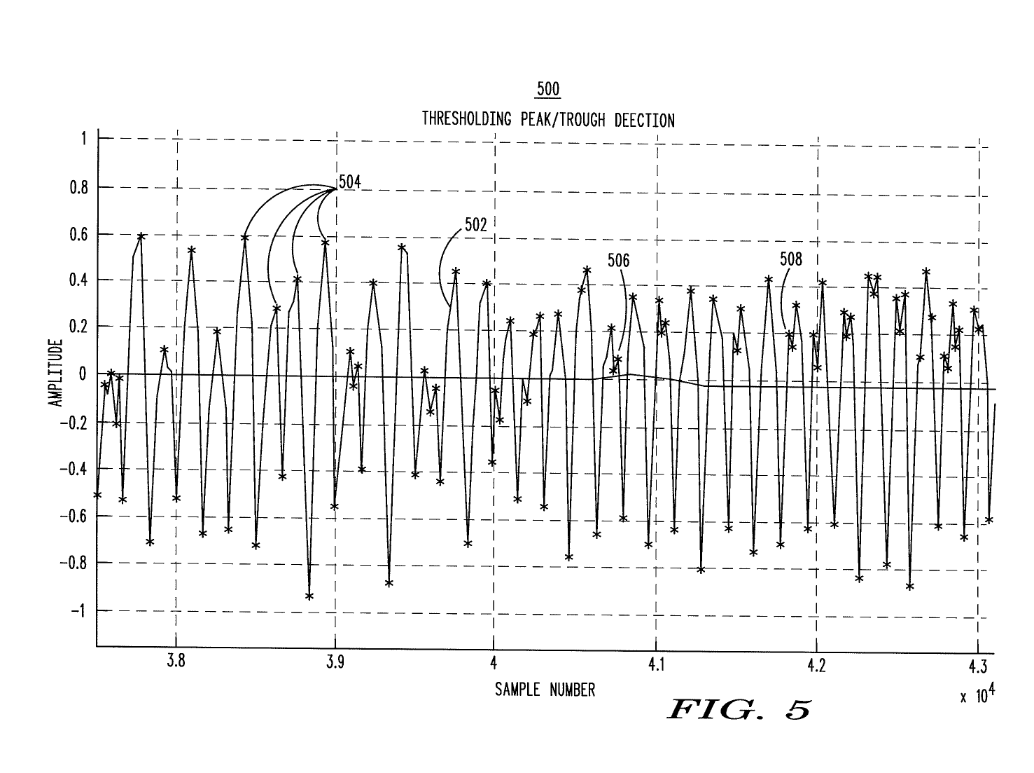

A third peak detector 330 works in a different manner. The third peak detector 330 has a discrete differentiator 332 followed by a zero crossing detector 334. There are two types of zero crossings transitions: from positive to negative and vice versa and ideally there should be one of each type per heart beat. The two types of zero crossings can be used as two separate identifiers of heart beats. Even after the filtering described above there may be small amplitude fluctuations in the signal that could potentially lead to false peaks being detected by a peak detector based on differentiation followed by zero-crossing detection. The potential for false peak detection can be understood by referring to FIG. 5. FIG. 5 is a graph 500 including a plot of a filtered Photoplethysmography signal 502 along with points 504 (only a few of which are numbered to avoid crowding) identifying peaks associated with individual heart beats that have been detected by a peak detector. Note there are a number of small amplitude fluctuations, e.g., 506, 508 that are not true signal peaks but could be misconstrued as such by a peak detector based on differentiation followed by zero-crossing detection. To address this issue of false peak detection, the third peak detector 330 suitably applies a criteria that requires that successive peaks be spaced by a certain number of samples (equivalent to a time increment). The required spacing can be fixed or varied for example as a function of a latest detected heart rate.

Although the system 300 as described above includes three particular peak detectors 326, 328, 330. In practice other known or yet to be developed peak detectors can be used in lieu or in addition to those described above.

The three peak detectors 328, 330, 332 are coupled to a counter 336. The counter 336 counts the number of counts identified by the peak detectors 328, 330, 332 in each window demarcated by the windower 324. The counter 336 is coupled to an averager 338 which generates an average peak count by averaging the counts of peaks detected by the three peak detectors 328, 330, 332. Averaging leads to a more reliable peak count. A frequency domain peak detector such as a peak detector that detects a peak in a power spectrum or a peak in an output of a Fast Fourier Transform (FFT) could also be used. Such frequency domain peak detectors involve a high computational cost or hardware complexity but do not require their output to be processed by the counter 336.

The averager 338 is coupled to a first-in-first-out (FIFO) buffer 340. At any given instant in time the FIFO buffer 340 holds a number (e.g., 32) of average peak counts of successive windows demarcated by the windower 324.

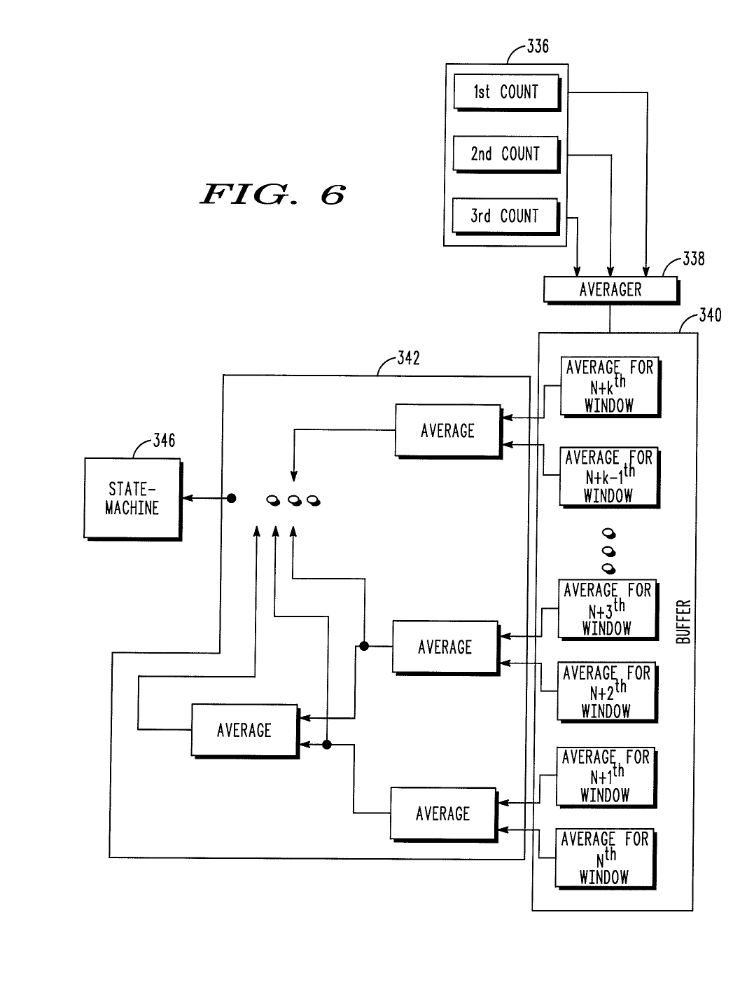

The contents of the FIFO buffer 340 are coupled out in parallel to a windowed averager 342. The windowed averager 342 computes a hierarchical set of averages of the contents of the FIFO buffer 340 spanning different time scales and a starting at different registers (correspond to different time indexes) in the FIFO buffer 340. By way of illustration assuming that the FIFO buffer holds N averaged peak counts for N successive windows that have been demarcated by the windower 324, the windowed averager 342 will computer 1 average over all N FIFO buffer registers, 2 averages over N/2 FIFO buffer registers (including one average over the first N/2 buffer registers and one average over the last N/2 buffer registers) and continuing in this pattern down N/2 averages at the smallest averagable times scale of 2 buffer registers. FIG. 6 is a more detailed view of a portion of the heart rate detecting system shown in FIG. 3 including the counter 336, the averager 338, the FIFO buffer 340 and the windowed averager 342. The windowed averager 342 is not shown in full detail, rather a dot-dot-dot notation indicates that the remainder of the topology of the windowed averager 342 follows the same pattern as the portion shown. As shown in FIG. 6 the averages from a shorter time scale (e.g., spanning two FIFO buffer registers) can be used as input to compute the averages for a next longer time scale (e.g., spanning four buffer registers).

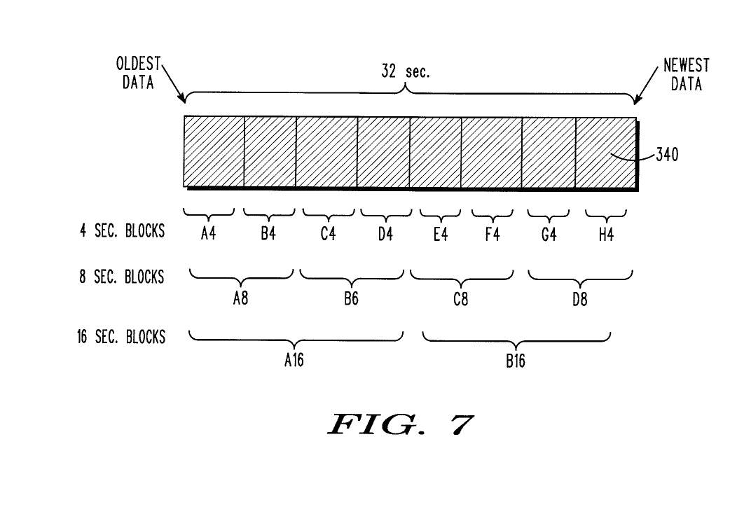

FIG. 7 is a schematic representation showing how a set of counts stored in a FIFO buffer 340 shown in FIG. 7 are aggregated hierarchically by the windowed averager 340 of the system shown in FIG. 3 to produce a set of running averages over three different time scales. There are eight averages each spanning four seconds (four registers of the FIFO 340, assuming the windower 324 produces one-second signal windows) and labeled A4, B4 . . . H4, there are four windows each spanning eight seconds and there are two windows each spanning sixteen seconds.

The FIFO buffer 340 is also coupled to a median selector 344 which selects the value in the FIFO buffer that is closes to the median. Alternatively, in lieu of the median another value such as the mode (most frequent value) or the average is used.

The FIFO buffer 340, the windowed averager 342 and the median selector 344 are coupled to a state-machine 346. The state-machine 346 implements a heuristic set of rules in order to output a heart rate estimate based on the contents of the FIFO buffer 340, averages produced by the windowed averager 342 and the output of the median selector 344.

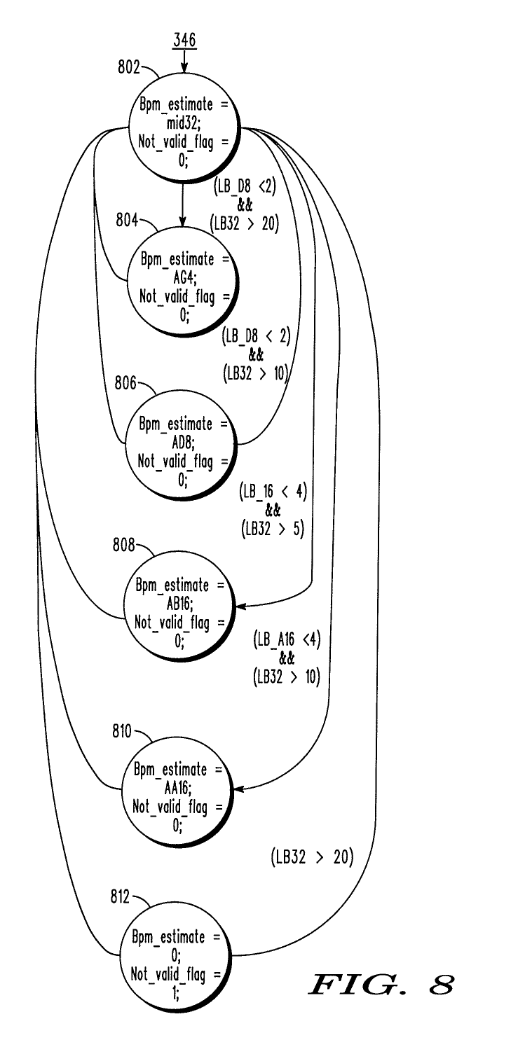

FIG. 8 is a block diagram of the state machine 346 of the system 300. The state machine 346 is initialized in a first state 802 in which the median value is output as the heart rate estimate and a “Not_valid_flag” flag is set to zero. The median value is denoted “mid32” in FIG. 8. Setting the Not_valid_flag to zero means that the heart rate estimate that is output is considered valid. The Not_valid_flag may be of used by parts of a larger systems that includes the heart rate sensing system 300. For example the Not_valid_flag can be used to control whether or not the heart rate estimate is displayed on a display.

There are Boolean expression rules that govern the transitions from the first state 802 to other states of the state machine 346. These Boolean expression rules are suitably evaluated for each new window period demarcated by the windower 324, after the FIFO buffer 340 has been updated. According to some embodiments the rules depend on counts of registers within a particular range of registers in the FIFO buffer 340 that contain counts that are considered invalid because the heart falls outside of prescribed limit. In certain embodiments the prescribed limit is a lower bound on the heart rate that must not be violated. According to one embodiment the lower bound is 45 beats per minute. In FIG. 8 such rules are expressed in a notation including a prefix LB followed by a suffix identifying a particular set of registers of the FIFO buffer 340 over which the count of registers containing invalid heart rates is made. The suffixes correspond to the codes used in FIG. 7 to identify subsets of the registers of the FIFO buffer 340. A special notation case LB32 specifies the count of registers containing invalid heart beats in the entire 32 register long FIFO buffer 340. In FIG. 8 two ampersands in expressions of the state-to-state transition rules represents the Boolean AND operation. Some states of the state machine 346 output estimated heart rates that are averages over one of the subsets of registers of the buffer. In FIG. 8 these averages are denoted with a prefix letter “A” followed by a suffix identifying one of the subsets of registers identified in FIG. 7. After the output associated with a state has been generated the state machine 342 returns to the first state 802.

The rule governing the transition from the first state 802 to a second state 804 requires that FIFO buffer 340 registers in the G4 subset (of which there are four) have less than two counts that violate the prescribed limit and that there be more than twenty registers that contain invalid counts in the entire FIFO buffer 340. The second state 804 will output the average heart rate AG4 over the set of registers designated G4 and will set the Not_valid_flag to zero, meaning the heart rate is valid.

The rule governing the transition from the first state 802 to a third state 806 requires that the FIFO buffer 340 registers in the D8 subset (of which there are 8) have less than two counts that violate the prescribed limit and that there be more than ten registers that contain invalid counts in the entire FIFO buffer 340. The third state 806 will output the average heart rate AD8 over the set of registers designated D8 and will set the Not_valid_flag to zero.

The rule governing the transition from the first state 802 to a fourth state 808 requires that the FIFO buffer 340 registers in the B16 subset (of which there are 16) have less than four counts that violate the prescribed limit and that there be more than five registers that contain invalid counts in the entire FIFO buffer 340. The fourth state 808 will output the average heart rate AB16 over the set of registers designated B16 and will set the Not_valid_flag to zero.

The rule governing the transition from the first state 802 to a fifth state 810 requires that the FIFO buffer 340 registers in the A16 subset (of which there are 16) have less than four counts that violate the prescribed limit and that there be more than ten registers that contain invalid counts in the entire FIFO buffer 340. The fifth state 810 will output the average heart rate AA16 over the set of registers designated A16 and will set the Not_valid_flag to zero.

The rule governing the transition from the first state 802 to a sixth state 812 requires that there be more than twenty registers that contain invalid counts in the entire FIFO buffer 340. The sixth state 812 will output a heart rate of zero and will set the Not_valid_flag to one, meaning the heart rate is invalid.

Although described hereinabove in the context of the heart rate sensing system, the state machine 346 can be used to process other types of systems and is particularly useful in connection with processing signals from sensors that are prone to signal dropout and/or faulty signals.

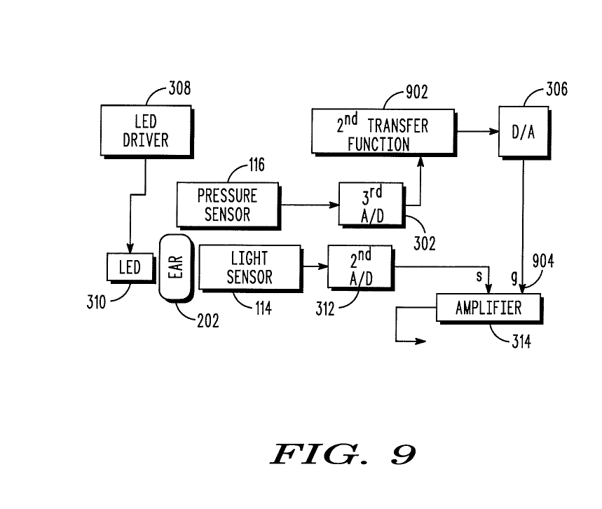

FIG. 9 is partial functional block diagram of a part of the system 300 shown in FIG. 3 according to an alternative embodiment of the invention. In the alternative shown in FIG. 9 a signal derived from the pressure sensor 116 is used to control the gain of the amplifier 314. In particular a digitized version of the pressure sensor signal is coupled through a second transfer function (or time domain filter) 902 and the D/A 302 to a gain control input 904 of the amplifier 314. Varying the gain in accordance with the signal derived from the pressure sensor 116 serves to compensate for fluctuations of the light sensor reading that are induced by movement of the wearer of the ear cuff 100 or other device including the system 300.

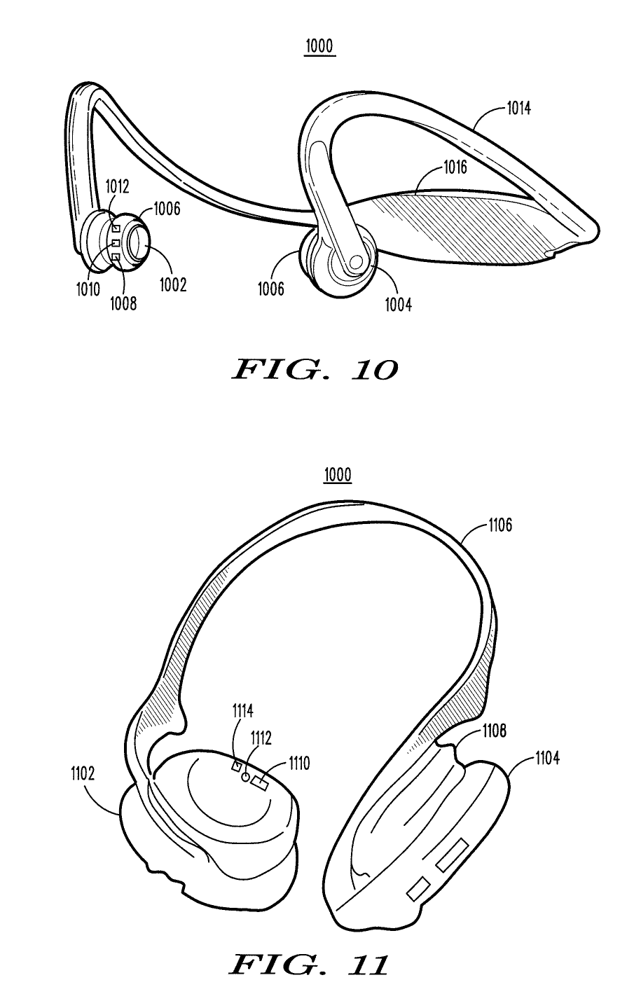

FIG. 10 is an audio headset 1000 that includes an optical heart rate sensing system (e.g., 300) according to an embodiment of the invention. The audio headset 1000 includes a pair of ear buds including a first ear bud 1002 and a second ear bud 1004. The ear buds 1002, 1004 include an outer annular foam pad 1006 that is adapted to fit in a person's ear. The foam pad is compressible, comformable and resilient. An LED light source 1008, a light sensor (e.g. photodiode) 1010, and a pressure sensor 1012 are fitted into the outer annular foam pad 1006 of the first ear bud 1002. A loop shaped harness 1014 connects the ear buds 1002, 1004. An enlarged cross section central portion 1016 of the loop shaped harness 1014 encloses a circuit board, or flex circuit (not shown) on which the circuits embodying a heart rate sensing system (e.g., 300) are implemented.

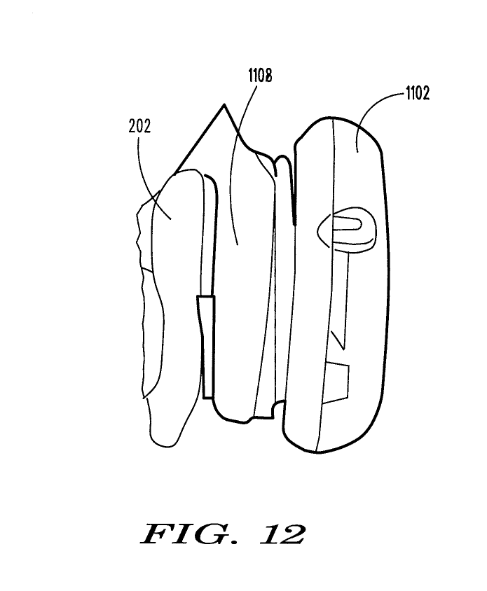

FIG. 11 is a set of headphones 1100 that includes an optical heart rate sensing system (e.g., 300) according to an embodiment of the invention. The set of headphones 1100 include a first headphone 1102 and a second headphone 1104 connected by a resilient “U” shaped harness 1106. Each of the headphones 1102, 1104 includes an annular foam pad 1108 that in use may be located on or surrounding a person's ears. An LED light source 1110, a light sensor (e.g. photodiode) 1012, and a pressure sensor 1014 are fitted into the annular foam pad 1008 of the first headphone 1002. The pressure sensor 1014 is located such that it will be in contact with the wearer's body when the headphones 1100 are being used. FIG. 12 shows one of the headphones 1102, 1104 shown in FIG. 11 located proximate a user's ear.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. A heart rate sensing system comprising:
a primary sensor for sensing a physiological phenomenon that is correlated with beating of an organism's heart and producing a first signal therefrom;
a pressure sensor for sensing a fluctuating pressure signal that is correlated with a noise signal component that is also sensed by the primary sensor;
an adaptive noise canceller coupled to the primary sensor and the pressure sensor wherein said adaptive noise canceller is adapted to reduce the noise signal component in said first signal and output a reduced noise output signal.

2. The heart rate sensing system according to claim 1 further comprising a bandpass filter coupled to said adaptive noise canceller, wherein said bandpass filter is adapted to filter said reduced noise output signal and output a signal with further reduced noise.

3. The heart rate sensing system according to claim 1 wherein said primary sensor is a light sensor.

4. A heart rate sensing system comprising;
a primary sensor for sensing a physiological phenomenon that is correlated with beating of an organism's heart and producing a signal;
a first peak detector coupled to said primary sensor;
a counter coupled to, at least, said first peak detector wherein said counter is adapted to count a first count of peaks detected by said first peak detector within each of a first series of windows;
a buffer coupled to said counter for receiving peak counts from said counter, wherein said buffer is adapted to store a plurality of counts at any given instant;
a processing circuit coupled to said buffer wherein said processing circuit is adapted to process said plurality of counts stored in said buffer and produce a heart rate estimate there from.

5. The heart rate sensing system according to claim 4 wherein said processing circuit comprises:
a windowed averager coupled to said buffer wherein said windowed averager is adapted to compute at least two different averages of said counts stored in said buffer wherein said two different averages are taken over two distinct sets of said plurality of counts;
a state machine coupled to said buffer wherein said state machine is adapted to receive said at least two different averages and process said at least two different averages and produce said heart rate estimate there from.

6. The heart rate sensing system according to claim 5 wherein said state machine comprises a plurality of output states each of which outputs one of said at least two different averages and a plurality of transitions to said plurality of output states.

7. The heart rate sensing system according to claim 6 wherein said plurality of transitions depend on inequality tests that compare at least one of said counts in said buffer to at least one predetermined limit.

8. The heart rate sensing system according to claim 7 wherein at least one of said inequality tests requires that at least a first pre-programmed number of counts in said buffer satisfy a first limit of said at least one predetermined limit.

9. The heart rate sensing system according to claim 8 wherein at least one of said inequality tests requires that at least a second pre-programmed number of counts in said buffer violate a second limit of said at least one predetermined limit.

10. The heart rate sensing system according to claim 9 wherein said first limit is equal to said second limit.

11. The heart rate sensing system according to claim 6 further comprising:
a second peak detector; and
wherein said counter is also coupled to said second peak detector wherein said counter is also adapted to determine a second count of peaks detected by said second peak detector within each of a second series of windows;
an averager coupled to said counter wherein said averager is adapted to compute a series of average peak counts, wherein each average peak count includes at least one of said first count corresponding to one of said first series of windows and one of said second count corresponding to one of said second series of windows, and wherein said peak counts received by said buffer comprise said average peak counts.

12. The heart rate sensing system according to claim 11 wherein said first series of windows is coincident with said second series of windows.

13. The heart rate sensing system according to claim 11 wherein:
said first peak detector is adapted to check that a first signal sample is less than a second signal sample and that said second signal sample is greater than a third signal sample and that a first quantity that is a function of, at least said second signal sample, is greater than a first predetermined threshold.

14. The heart rate sensing system according to claim 13 wherein:
said second peak detector comprises a differentiator followed by a zero-crossing detector.

15. The heart rate sensing system according to claim 13 wherein:
said second peak detector is adapted to check that a second quantity that is a function of, at least said second signal sample, is greater than a second predetermined threshold.

16. A heart rate sensing system comprising:
an optical detection subsystem comprising an optical source for illuminating a body part with optical radiation, and an optical sensor for detecting said optical radiation after said optical radiation has been modulated by a physiological phenomenon occurring in said body part that is correlated with a heart beat, wherein said optical sensor produces a first signal;
a secondary sensor for sensing a second signal that is correlated with a noise component of said first signal, wherein said second signal is coupled into said optical detection subsystem in such manner as to effect an amplitude of said first signal.

17. The heart rate sensing system according to claim 16 wherein said secondary sensor comprises a pressure sensor adapted to measure pressure against said body part proximate said optical sensor.

18. The heart rate sensing system according to claim 16 wherein:
said optical detection subsystem comprises an optical source driver and wherein said second signal is used to control a power level at which said optical source driver powers said optical source.

19. The heart rate sensing system according to claim 18 wherein said secondary sensor comprises a pressure sensor adapted to measure pressure against said body part proximate said optical sensor.

20. The heart rate sensing system according to claim 16 wherein
said optical detection subsystem comprises an amplifier that amplifies said first signal and wherein said second signal is used to control a gain setting of said amplifier.

21. The heart rate sensing system according to claim 20 wherein said secondary sensor comprises a pressure sensor adapted to measure pressure against said body part proximate said optical sensor.

22. A heart rate sensing apparatus comprising:
a body of compressible material have a surface adapted to be placed against a body part;
a optical radiation source mounted on said body of compressible material; and
an optical radiation sensor mounted on said body of compressible material.

23. The heart rate sensing apparatus according to claim 22 further comprising an ear cuff to which said body of compressible material is attached wherein said ear cuff is adapted to hold said surface against said body part.

24. The heart rate sensing apparatus according to claim 22 wherein said body of compressible material is attached to a headphone.

25. The heart rate sensing apparatus according to claim 22 wherein said body of compressible material is attached to an ear bud of a headset.

26. The heart rate sensing apparatus according to claim 22 further comprising a pressure sensor disposed at said surface and adapted to measure a pressure between said body of compressible material and said body part.

27. A sensing system comprising;
a sensor for sensing a phenomenon and producing a signal;
a buffer coupled to said sensor wherein said buffer receives a sequence of values derived from said signal and wherein said buffer is adapted to store a plurality of values at any given instant;
a windowed averager coupled to said buffer wherein said windowed averager is adapted to compute at least two different averages of said values stored in said buffer wherein said two different averages are taken over two distinct sets of values stored in said buffer;
a state machine coupled to said buffer wherein said state machine is adapted to receive said at least two different averages and process said at least two different averages and produce a signal estimate there from.

28. The sensing system according to claim 27 wherein said state machine comprises: a plurality of output states each of which outputs one of said at least two different averages; and a plurality of transitions to said plurality of output states.

29. The sensing system according to claim 28 wherein said plurality of transitions depend on inequality tests that compare at least one of said values in said buffer to at least one valid signal limit.

30. The sensing system according to claim 29 wherein at least one of said inequality tests require that at least a first pre-programmed number of values in said buffer satisfy a first limit of said at least one valid signal limit.

31. The sensing system according to claim 30 wherein at least one of said inequality tests require that at least a second pre-programmed number of values in said buffer violate a second limit of said at least one valid signal limit.

32. The sensing system according to claim 31 wherein said first limit is equal to said second limit.

33. The sensing system according to claim 27 further comprising:
a peak detector coupled to said sensor;
a peak counter coupled to said peak detector and wherein said peak detector is coupled to said buffer and wherein said values derived from said signal are counts output by said peak counter.

34. The sensing system according to claim 33 wherein said sensor is a heart rate sensor.
